(19) United States
(12) Patent Application Publication
(10) Pub. No.: US 2004/0138539 A1
(43) Pub. Date: Jul. 15, 2004

(54) Non-invasive blood monitor

(76) Inventors: Paul R. Jay, Stittsville (CA); Zeljko Ribaric, Kanata (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(21) Appl. No.: 10/752,012

(22) Filed: Jan. 7, 2004

Related U.S. Application Data
(60) Provisional application No. 60/438,306, filed on Jan. 7, 2003.

(51) Int. Cl. A61B 5/00

(52) U.S. Cl. 600/322; 600/316

(57) ABSTRACT

A blood species concentration tester, and corresponding method, are disclosed herein. The tester employs the measurement of the characteristics of at least one wavelength of light returned from the eye of a patient to determine the concentration of a species, such as glucose, in a blood stream. The correlation of the characteristics of the at least one wavelength with each other, is utilised to derive an accurate measure that can provide a simpler and more accurate test of blood sugar levels, along with other species concentration levels.

### CROSS REFERENCE TO RELATED APPLICATIONS

This application claims the benefit of U.S. Provisional Patent Application No. 60/438, 306, filed Jan. 7, 2003, which is incorporated herein by reference in its entirety.

### FIELD OF THE INVENTION

The present invention relates generally to detecting the presence of species in blood. More particularly, the present invention relates to non-invasive measurement of the levels of species, such as glucose, dissolved in blood.

### BACKGROUND OF THE INVENTION

According to 1999 figures, some 16,000,000 people in the United States, and over 100,000,000 people worldwide, have been diagnosed with diabetes. Frequent self monitoring of blood glucose is crucial for the effective management of the disease to allow for treatment and reduction of the mortality associated with diabetes. Self monitoring of glucose levels typically involves testing of either urine or blood. Typically, urine testing is considered to be less accurate than an analysis of the glucose levels in blood, and at best it provides an indication of the blood sugar levels at a previous point in time. Determination of blood sugar levels through an analysis of the blood itself, is by definition invasive, as it requires the extraction of blood from the subject.

Glucose is measured to allow a diabetic to determine the present levels of sugar in the blood stream, which is an indicator of the insulin levels. If blood sugar levels are outside of the normal band, they can be controlled through the administration of insulin. However, as a result of the lack of consistent testing, many diabetics are unable to properly determine the glucose levels in their blood stream, and thus cannot properly determine the amount of insulin required to maintain blood sugar levels that are consistently in an acceptable range.

Ideally diabetics test blood samples four to seven times per day. Due, at least partially, to the invasive nature of the testing, the average diabetic performs a blood glucose level test less than twice a day. The primary reason provided for the lack of testing relates to the general unpleasantness (pain and inconvenience) of the current blood glucose tests (fingerstick), which require the breaking of the skin on a finger tip. After breaking the skin, a small amount of blood is collected through massaging of the finger near the wound, so that a drop of blood can be smeared on a test strip. The strip is then handled to wipe off excess blood, and is then provided to a glucose tester. After use, the strip is discarded. These disposable strips are essentially a consumable, and must be replaced at a cost to the diabetic. The recurring cost serves as a disincentive for some patients to regularly test.

The puncturing is unpleasant for many patients, and is physically difficult for patients with very low blood flow in the extremities, a condition common among many diabetics especially the elderly. Additionally, haemophiliacs are reluctant to intentionally cause bleeding because of the difficulties associated with stopping the bleeding. Individuals on blood thinners are equally reluctant to intentionally cause bleeding for the same reason.

Testing of blood glucose levels through conventional techniques involves piercing the skin of a finger, or thumb, and extracting, from the wound, a small blood sample. For the analysis of this sample to be accurate, the blood must not be contaminated. As a result, diabetics are typically required to wash their hands thoroughly to ensure that no food matter is present in the hands, as the food matter will skew the blood glucose levels. Additionally, other contaminants that may not skew the glucose levels reported by the tester should nevertheless be removed from the hands to ensure that contamination of the blood stream does not occur, especially since poor circulation in the extremities of a diabetic is common and the combination of a contaminant and poor circulation can result in infection.

At the start of 2003, there was only one FDA-approved noninvasive glucose monitor on the market—the GlucoWatch automatic glucose biographer by Cygnus Inc. This device tests glucose levels in the blood through intact skin by utilising an amperometric biosensor. This equipment is very expensive, and requires a great deal of work to reduce its size.

Currently the techniques known in the field of blood glucose level monitoring include Subcutaneous testing, Microdialysis, Wick Extraction, Implanted Electrochemical Sensors, Implanted Fluorescence Sensors, Dermal, Epidermal, Infrared Spectroscopy, and combinations of the above listed. Extraction Fluid Techniques including lontophoresis, Skin Suction, and Suction Effusion are also known, as are optical techniques, such as Near Infrared Spectroscopy (NIR), Infrared Spectroscopy (IR), Raman Spectroscopy, Photoacoustic Spectroscopy, Scatter Changes, and Polarization Changes. Typically these techniques are used to determine the blood glucose levels by testing one of the following fluids: Blood, Interstitial fluid, Ocular fluids, Tears, the Aqueous humor, sweat, saliva and urine. As discussed above, urine based testing, as well as testing saliva and sweat, are not considered to be accurate indicators of the present blood sugar levels. There are also known safety issues associated with testing the Aqueous humor. Other techniques are currently being researched including testing of finger tips, the cuticles, the web between fingers, the forearm, ear lobe, inner lip, abdomen and eye.

The objective of many researchers in the field is to produce a technique to allow a patient to self-test blood sugar levels in a more convenient manner. Some of the efforts in this field have been aimed at limiting the amount of handling that a blood teststrip requires. As a result of such efforts some blood glucose testing equipment stores a drum of test strips, and ejects one at a time, so that the diabetic does not need to handle individual strips. Though this reduces the amount of handling associated with each strip it is still an invasive technique.

The problem at the heart of glucose testing, as it is in the testing of other dissolved species, is one of determining an accurate estimate of the concentration of a particular substance (e.g. glucose) in a patient's blood, without having to draw blood from the patient or having to introduce any device for permanent implantation into the patient's body.

It is known that glucose levels in the blood stream can be determined through optical techniques. By passing controlled optical energy through a sample of blood, and measuring the optical energy after it has passed through the sample, glucose levels can be determined through measurement of the polarisation of the light, or through the absorption of particular frequencies of the transmitted light. Alternatively the amount of light passed through the sample can be correlated to blood sugar levels, as glucose has known optical properties such as defined reflectivity and absorption characteristics. These techniques can also be applied to determining the concentration of other components carried with the blood, such as, alcohol, cholesterol, and drug by-products at any given time.

For diabetic patients the most widely-used solution still involves a finger-prick or other means to withdraw blood for analysis. Recent improvements have greatly simplified this process, but it is still invasive, and to many patients it is painful (especially the very young and the elderly) as well as inconvenient. As a result, many patients avoid the required frequency of blood measurements, but by so doing undermine the quality of their care, with potentially serious consequences, especially in the longer-term.

The process for determining the glucose concentration from the sample extracted is subject to how accurately the procedure is implemented, and this can contribute to incorrect readings.

Approvals are in progress for devices that use near-surface Infra-red spectroscopy on the skin to provide an estimate of the glucose concentration, however the accuracy of the readings is a major challenge owing to the poor transmissivity of the skin to infra-red light, and multiple scattering by the skin, which is also subject to considerable variations according to degree of hydration or temporal changes in skin thickness.

Another approach uses the infrared reflectance of the inner lip, which seems to correlate well with blood glucose concentration but with a 10 minutes delay.

Another approach being evaluated involves implanting a device that will both measure the glucose level and then activate an implanted source of insulin to be released as required. Although this would provide a virtually continuous solution to the problem, it has the disadvantage of requiring surgery for the implantation, and periodically for replacement of exhausted devices.

An article by McNichols, Cameron and Cote (Texas A&M University) describes the transmission of a light beam across the front surface of the eyeball (just behind the cornea) to determine the glucose level based on polarisation variation in the aqueous humor of the eye. However, the disclosed this technique is prone to poor specificity, and involves a time lag from the concentration present in the blood to the achieving of a proportional concentration in the aqueous humor. The technique is also sensitive to rotations introduced by substances such as ascorbate and albumin, both of which can be present in the humor. An advantage of the polarisation technique is that wavelength specificity is less of an issue.

Another approach (eg Tarr and Steffes, Georgia Tech) proposes an adaptation of stimulated Raman Spectroscopy using a beam passed tangentially through the front of the eye to probe the spectroscopic characteristics of the aqueous humor. This is an elegant approach but involves more costly optics and a setup that could be difficult to reproduce in a portable consumer-level machine.

It is, therefore, desirable to provide a method of testing blood component levels to allow determination of the levels while minimising the inconvenience of the test to the patient.

### SUMMARY OF THE INVENTION

It is an object of the present invention to obviate or mitigate at least one disadvantage of previous blood component level testing methods.

In a first aspect of the present invention there is provided a blood species tester for determining the concentration of a species in a blood stream of a subject. The tester comprises a light source, a light detector and a controller. The light source transmits a light pulse, having at least one known characteristic, to an eye of the subject. The light detector receives and characterized a modified light pulse. The controller determines a transfer function relating at least one of the at least one known characteristics to at least one characteristic of the modified pulse, and maps the determined transfer function to a species concentration in accordance with a predetermined mapping function.

In an embodiment of the first aspect of the present invention, the species is selected from a list including glucose, alcohol, and tetrahydrocannabinol. In another embodiment, the at least one known characteristic is selected from a list including wavelength and intensity and the at least one characteristic of the modified pulse is selected from a list including intensity, polarisation, and fluorescence. In another embodiment, the light source includes means for transmitting a pulse having a plurality of known characteristics, where the means for transmitting a plurality of known characteristics may include a plurality of light sources, each of the plurality for transmitting a light pulse, having at least one known characteristic, and wherein the controller determines transfer functions relating each of the known characteristics to measured characteristics in the modified pulse. In another embodiment of the present invention, the tester includes a memory means for storing historical species concentration information, and preferably the controller includes subject identification means for obtaining subject identification information from at least one characteristic in the modified light pulse, for storing identification information with associated historical species concentration information in the memory means, and for differentiating species concentration information stored in the memory on the basis of the associated identification information. In another embodiment the tester further includes a calibration mechanism for determining the mapping function by correlating a plurality of determined transfer functions to species concentrations obtained using an external species concentration tester.

In another embodiment there is provided a method of determining a species concentration in a subject's bloodstream. The method comprises three steps. The first step is the generation and transmission of a light pulse having at least one known characteristic. The second step is the receipt and characterization of a modified, returned light pulse. The third step is the determination of the species concentration in accordance with a mapping function and a transfer function. The transfer function relates the at least one known characteristic with a corresponding characteristic in the modified, returned light pulse.

In an embodiment of the second aspect of the present invention, the method includes the steps of calibrating the mapping function to map the transfer function to known species concentrations in advance of the step of determining the species concentration. In another embodiment of the second aspect of the present invention, the step of generating and transmitting includes transmitting the light pulse into an eye of the subject and the step of receiving and characterizing includes receiving the modified returned light pulse from the ocular vasculature. In another embodiment, the step of generating and transmitting includes transmitting a light pulse having a plurality of known characteristics. In a further embodiment the step of generating and transmitting includes transmitting a series of light pulses, where optionally each of the series of light pulses has at least one known characteristic, at least two of the series of light pulses have different known characteristics. Furthermore, the step of receiving and characterizing optionally includes receiving and characterizing a plurality of modified returned light pulses, and the step of determining the species concentration includes determining a transfer function relating the known characteristic of each light pulse to a corresponding characteristic in a corresponding returned modified pulse. A further embodiment of the present invention includes determining the transfer function in accordance with external environmental factors such as ambient temperature. In a further embodiment, there is included the further step of recording the determined species concentration in an archive of historical species concentrations.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic illustration of a testing apparatus of the present invention;

FIG. 2 is a schematic illustration of an alternate testing apparatus of the present invention;

FIG. 3 is a flowchart illustrating a method of the present invention; and

FIG. 4 is a flowchart illustrating a further method of the present invention.

### DETAILED DESCRIPTION

Generally, the present invention provides a method and system for noninvasive determination of blood component levels. More particularly, the present invention provides a method for measuring blood component levels by using the optical characteristics of the component in the blood stream when subjected to at least one wavelength of incident light.

The present invention represents an improvement on the current approaches to measuring levels of components or species present in the blood, such as blood sugar (glucose) for patients with diabetes. The approach of the present invention uses a pulse of light introduced into the eye by means of a device, which is preferably handheld, and performs measurements on the returned light. The measurements of the returned light are processed to give a reading of the concentration of a species in the blood. Many given species have known optical characteristics when dissolved in the bloodstream. These optical characteristics are used, in conjunction with the calibrated device, to map a transfer function, relating the input light to the returned light, to a concentration level. Thus, in one example, the species is known to absorb light of a given wavelength, the degree of the absorption being a function of the concentration of the species, thus the degree of absorption is used to determine the concentration of the dissolved species. One skilled in the art will appreciate that the optical characteristics of the species are used to determine the levels of the species required to alter the incident light in such a way that the result matches the light returned.

As it is an optical technique that relies upon reflectivity of incident light, the present method is a non-invasive method that can be fast, painless and convenient. As a result, glucose monitoring can be simplified and made less odious, which will encourage more frequent testing that can help provide adequate control and greatly reduce the complications seen in these patients and consequently reduce health care costs.

An embodiment of the present invention is a new portable optical device for monitoring blood glucose. It can measure the blood glucose level in the eye and preferably makes use of the direct optical access to the vasculature through the retina (more specifically the choroid). The tester of the present invention can combine multiple types of optical measurements to factor out physiological and environmental factors that reduce the accuracy and reliability of a single optical measurement. The device transmits a pulse of light having known characteristics selected to correspond to known optical properties of the dissolved species. The transmitted light is returned to the device, by mechanisms such as reflectivity and scattering. However, the returned light is modified as it passes through capillaries in front of the reflective back layer of the eye. The returned light pulse is characterized by the tester of the present invention to determine at least one relevant transfer function that can be correlated to the species concentration.

The sensitivity of optical measurements (reflectance, fluorescence, Raman Spectroscopy, etc) to glucose concentration has been demonstrated and methodologies depending on these optical measurements have been proposed as non-invasive techniques to monitor blood glucose. Most of the techniques have concentrated on measurements of blood glucose through the skin (in different areas of the human body). Most require complex multivariate calibrations that make the test difficult to perform. Furthermore they are very susceptible to confounding factors such cleanliness, skin condition, temperature, skin tone, etc.

A few techniques have been proposed that measure ocular optical properties (cornea, vitreous, lens, aqueous) but none takes advantage of the access to the vasculature in the retina. All depend on only one type of optical measurement and are susceptible to physiological factors, as are skin measurements. Furthermore, they are either, difficult and expensive to perform (i.e. Raman spectroscopy), or unreliable (polarization, reflectance). Finally, unlike the vasculature which responds within seconds or minutes to glucose level changes, other ocular tissues are slow (on the order of hours) to reflect blood glucose changes. These delays are much too long for these measurements to be reliable for blood glucose control. The calibration the tester of the present invention can make use of the differing optical properties of the dissolved species due to access to the vasculature. This allows a calibration process against a known conventional test, such as a blood test, to determine data points along the mapping function that correlates the transfer function to the species concentration. Known techniques of interpolation between the accumulated data points can be used to determine the rest of the mapping curve. By avoiding the reliance upon expensive techniques such as Raman spectroscopy, the tester of the present invention can be made so that it can be calibrated to each patient. Thus, each device can be calibrated to an individual patient using a conventional test, and can use a series of different optical properties of the desired species to set characteristics to test against. This allows the tester to overcome masking of the desired species by another species present in the bloodstream, though the use of multiple tests. The sensitivity of the vasculature allows for reproducibility of results, and rapid response time.

Blood glucose concentration measurements are critical to the management of diabetes. For proper management of the disease it is suggested that patient test their glucose level up to seven times a day. Frequent measurement is essential to avoid large fluctuations in the levels of glucose that are believed to be extremely damaging to sensitive tissues such as the retina. Access to the accurate, non-invasive test of the present invention allows patients to perform the test frequently without having to draw blood or use a consumable element such as a testing strip.

The novel technique of the present invention has the potential to improve the quality of life (by simplifying the control of the glucose levels) and the health (more frequent measurements correlate to more even control of glucose levels) of millions of diabetics. This is especially important given the potential for long life expectancy of patients suffering from this disease, if diabetes care is well-managed.

Embodiments of the present invention may have a significant impact on a number of other clinical populations. The optical properties can be adjusted to focus on the detection of many substances transported by the vasculature. For example: hormone levels for menopausal women, iron levels in anaemic patients, lithium level in manic/depressive patients, drug levels in numerous other patients, such as surgical candidates who are required to have a defined concentration of blood thinning drugs prior to surgery. The tester of the present invention offers a platform that can adapt to many other situations in the healthcare domain. Dissolved species such as alcohol and tetrahydrocannabinol (THC) have known effects in the vasculature, and thus can be tested for using the tester of the present invention. The present invention provides a test that is accurate, and is less susceptible to tampering than an exhalation based test, but is also less invasive than a blood test, and is thus easier to administer as a road-side test. The tester of the present invention may optionally include an integrated retinal scanner for obtaining subject identification information to associate and store with the species concentration level information. This subject identification information can be used to unambiguously associate the species concentration levels with a single subject, which has use in both medical and law enforcement applications. Other dissolved species, such oxygen and performance-enhancing drugs, can be tested for using a tester of the present invention. As noted above the presence of a drug ingested immediately before a test is not always detectable by a urine test, which leaves an avenue for the use of performance enhancing drugs in sport. The use of a non-invasive test allows for more immediate results than taking a body fluid sample and sending it to a lab for analysis. As the tester can be used to perform a plurality of tests, it can additionally be used to determine combinations of different dissolved species that can interact, thus in a medical setting, for example, a test can be quickly performed to ensure that prior to the administration of a given drug the patient does not have interacting drugs in the bloodstream.

The tester and method of the present invention rely upon comparison of changes in at least one of a plurality of optical properties of light returned back from an eye. The input light can be controlled to be time-varying in a known fashion, so that a series of returned light pulses can be analysed. The ability to use either a single or multipleinput light pulses allows for correlation of various factors including reflectance, scattering, fluorescence, and polarization resulting from species levels in the ocular vasculature, as well as the change in these factors in response to different wavelengths and intensities of light. Using a combination, ratio, or differences of two or more of the various measures as a transfer function, a blood glucose, or other such dissolved species, level can be predicted. Through the use of a controller in the tester a response operator curve for specificity and sensitivity can be calculated and used as a comparison benchmark for the measured readings. One skilled in the art will recognize that this methodology and testing equipment can be applied to determine levels of other species in the blood stream, either in combination with glucose or other species levels, or in isolation. Thus the tester of the present invention can be applied to determine levels of dissolved drugs, or alcohol, or of ingested or absorbed contaminants such as lead, mercury etc. The controller can preferably administer a signature matching technique to determine the presence of chemicals by matching the characteristics of the returned light to pre-programmed signatures stored in memory. Signature matching algorithms are known, but are typically not included in blood monitors and testers as a result of cost. However, due to the programmable nature of the tester of the present invention, a variety of different tests can be administered and processed without additional cost. Additionally, one skilled in the art will appreciate that the administration of multiple tests can be employed for the detection of multiple different dissolved species.

This is a field that represents considerable inconvenience and quality-of-life disruption for a very large proportion of the population, and also involves considerable cost to health services, patients and insurance providers. As a result there is intense activity to find an effective yet simple solution to the problem of measuring and controlling glucose levels in diabetic patients. The consequences of untreated or ineffectively treated diabetes can be severe, involving blindness, loss of extremities and early death.

The tester of the present invention provides patients with a simple, easy-to-use device that will provide virtually immediate readings of blood sugar level, and even offer related data such as comparison with the patient's variations of glucose level (according to time of day, activity level etc) and calculations to facilitate estimates of the insulin dosage required. In an envisaged embodiment, the tester is a portable hand-held device that would be as easy to use as currently available commercial ‘in the ear’ IR thermometers. One envisaged embodiment also benefits from the availability of a communicating interface that allows data measured to be transmitted to a local computer or remote database monitoring system. This feature provides the patient with more extensive monitoring resources and services to help optimize dose levels, choice of insulin products, and also provide valuable accurate statistics gathering for higher level health management improvements. One skilled in the art will appreciate that the tester of the present invention can provide analysis of the readings through a local display, as well as through an interface to a remote station. The local display can be a simple visual display, such as a liquid crystal display (LCD), or may optionally provide an interface for operators with visual impairments such as Braille or audio interfaces. The time based analysis of trends can also be provided through either the remote station or through the tester itself. The inclusion of a communication interface to a remote station, such as a standard personal computer, allows for a richer presentation of the data as well as providing a simple mechanism to transmit measurements to an offsite monitoring facility such as a hospital or other such medical establishment.

The same device principle can be extended to measure other properties of blood that normally require withdrawn samples (e.g. cholesterol measurement) or various other maladies that can be detected based on optical measurements made through the eye and accompanying vasculature. Many medical treatments can involve the use of medications, for which the ideal uptake by the body is difficult to assess, especially outside of the hospital environment. This technique could be tuned to detect certain species and raise alarms if the blood concentration falls below or rises above certain limits. This allows the method to be beneficially applied to patients or workers who may potentially be exposed to contaminant species such as lead, mercury, airborne pollutants or toxins (e.g. anthrax and botulism) that might be a threat to personal health or security. It is presently contemplated that an embodiment of the present invention may be provided as either an add-on to, or as an integral part of, a pair of glasses, so that either continuous or timed interval measurement of a determined species can be provided while not greatly impairing the subject's vision.

Considerable attention is being paid to health care techniques that could facilitate homecare surveillance and relieve in-patient facilities. The need for this type of improvement will grow with the increased aging demographics of North Americans (and other societies) over the coming 10-20 years.

In a presently preferred embodiment, the tester would comprise a light source, a focuser, such as a lens a microdiffractive array or a mirror, one or more optical detectors, a pair of wavelength filters, and a controller. The light source may be a broad-band source such as a superluminescent Light Emitting Diode (LED), which typically provides compact size and low cost, but could additionally be another light source such as a broadband LED, a laser, a tuneable laser, or the light source could be a combination of different types of light sources. A pair (or more) of optical detectors are preferably employed to sample returned signals at specific wavelengths determined by the controller to represent the factors relating to the species being tested, though one skilled in the art will appreciate that a single optical detector can be employed if the redundant detection is not required for a particular application. The wavelength filters may be Fiber Bragg Grating wavelength filters, or other such filters, selected so that the detectors provide accurate readings of returned light corresponding to the species line (e.g. glucose line) and a reference line (e.g. haemoglobin) from which the particular concentration can be computed. One or more of the detectors preferably detects a returned light signal for time-dependent analysis, such that the fluorescence of a particular signal, or another such signature for a particular species, can be used to identify that species and correlate it with measurements obtained by a different method, for maximum accuracy. The controller serves to manage the current and biasing of the light source as well as the photodetectors, and if necessary the temperature of other components, such as the Fiber Bragg Gratings or other such filters, to ensure stability of the wavelengths, and the temperature of the light source to ensure its stability. The controller is additionally employed to combine the necessary analog/digital converters (and vice versa) to detect the optical signals collected as well as to control the optical pulses emitted. The same controller may also manage the temperature stability of the source and gratings, and optionally collects and stores calibration data, recent readings and patient history, for comparison with latest readings. The controller also preferably offers communication capability ranging from transferring the data collected to a patient's personal computer, PDA or even to a remote medical centre by an appropriate interface adapter. Fitted with an appropriate interface, the device could also provide for automated alarm generation (and notification of emergency services) in the event that glucose count falls outside the allowable level for that patient. The automated alarm generation may optionally employ subject-set thresholds, so that a subject can set alarms indicating known conditions under which problems or particular physiological reactions have previously occurred. Thus, if a subject knows that shortly after determining that blood sugar has reached a particular threshold, an alarm can be set, to alert a remote station that the subject may need assistance. This enables potential benefits at a health service management level for widespread accurate data collection for patients or persons at risk of exposure to detectable species. It enables optimization of health services, improved understanding and control of epidemiology, and even provides potential for patient-focussed service opportunities relating to needs for consumption of certain types of medication products. One skilled in the art will appreciate that the communications interface to the tester can be implemented using standard components such as Bluetooth, cellular or IEEE 802.11 interfaces for wireless communications. The controller preferably can utilise the historical patient information stored in the memory of the tester to “learn” patient specifics such as iron content. These patient specifics can aid in the determination of the dissolved species levels, as they are typically fairly constant and can be modelled in terms of their interaction with the transmitted light. The learned patient specifics can also be used to identify the patient using background variables. Such a feature can identify that a particular patient typically has iron content, or another such background element, within a certain range, and if the reading is outside the range it can be flagged as an anomaly. This detection system can be used by medical practitioners to ensure that patients are not using family members or other such third parties to take measurements in an out patient situation.

In another embodiment, the tester of the present invention can use the background characteristics of a scan, or retinal information, to differentiate between a number of different subjects, that is, as subject identification information. This allows the tester of the present invention to record readings from a variety of different subjects, and to differentiate between the readings. This allows a single tester to be used by an entire family, and to record the readings from the different individuals in a manner that allows the stored readings to be differentiated from each other, so that medical analysis at a future point is still possible. In another application, a single tester can be used for a complete slate of athletes in a testing for performance-enhancing drugs, the retinal scan can serve as a sufficiently unique key to identify any athlete found to be using the performance enhancing drug. As noted above subject identification information may be used to unambiguously associate a subject with a species concentration level.

It is common for optical controllers to be used to control optical functions in devices, so that the optical function, or emitted signal, satisfies a desired transfer function with respect to an input signal. One skilled in the art will readily appreciate that the controller of the tester of the present invention would be employed to ensure that the light used as the input pulse to the eye is meeting the desired characteristics. Thus the controller knows the input to the eye. The light provided as input is then returned in part or reflected by the aqueous humor, or the ocular vasculature. The amount of light returned, and its characteristics, including the scatter pattern, the reflectivity at determined wavelengths, the polarisation, the fluorescence, and the overall amount of light returned are dependent upon the species concentration in the blood stream. Thus, by sampling the returned light, the tester can provide to the controller the output of a “black box”. Having both the input and output of a black box, the controller is able to determine a transfer function, and through the analysis of the transfer function in conjunction with the known properties of the species being examined, and preferably historical information about the patient, the concentration of the species, such as glucose, can be determined. However, it is known that a variety of different species are present in the blood stream, and a number of them may have similar effects on a particular characteristic. To compensate for this, the tester of the present invention can be used to determine a transfer function for at least two characteristics in the returned light and create a correlation used to determine a blood-glucose level, or other such dissolved species level. For example, a known set of wavelengths are provided as inputs in a time-varying manner by the light source. The particular wavelengths provided are known to have a distinct polarisation. The information about the wavelengths and their polarisation are stored by the controller. The returned light is then sampled by the sensors, and information relating to the amount of returned light, the overall reflectivity, and the amount of light returned at each of the wavelengths is provided to the controller. As different species in the blood stream have different absorption and reflectivity characteristics, the reflectivity values for the various wavelengths can be correlated to determine the blood-glucose levels. Additionally, each of the returned wavelengths is analysed to determine the polarisation of the reflected light. As various species are known to cause polarizations in different wavelengths, this set of information is also provided to the controller. As the controller is provided with a set of benchmark data that is used to determine how each of a number of species interact in the blood stream to affect reflectivity and polarisation, the transfer function relating the input and output light pulses is then used to determine the blood-glucose levels. One skilled in the art will appreciate that the polarization of certain wavelengths of the transmitted light introduces time delay for the polarized wavelengths. Thus, a suitably fast controller can time gate the returned light at different wavelengths, and use this information in both the identification of dissolved species, and in the determination of the dissolved species levels. One skilled in the art will appreciate that the calculation of more than one transfer function, corresponding to different characteristics of the dissolved species can be employed to provide redundancy as well as greater accuracy, as can the calculation of one transfer function relating a plurality of known characteristics of the transmitted pulse or pulses to a plurality of measured characteristics in the modified and returned pulse.

Calibration of a controller as described above is taught in co-pending applications, U.S. patent application Ser. No. 10/206,051 filed Jul. 29, 2002, PCT Serial No. CA03/01143, and Canadian Patent Application No. 2,436,177, all of which are expressly incorporated herein by reference.

As testers are typically not subject to wide temperature fluctuations or other such stress, it is not likely to experience much drift, however programmed routines within the controller allow for rapid recalibration. The recalibration preferably utilizes a conventional blood test administered at the same time as the optical test to provide a reference level. In one embodiment, the tester is calibrated in conjunction with a remote device, and is preferably done under physician supervision. In another embodiment, the tester includes a blood tester, and the subject simply administers both a blood test and an optical test to periodically confirm calibration of the tester. The latter embodiment allows for either frequent recalibration, or serves to determine that the device has drifted, and as a result requires recalibration under the supervision of a physician. In-the-field calibration, or calibration testing, can be accomplished using off-the-shelf components and provides for a well-calibrated device. The initial calibration, along with the period recalibrations, allows for component specific calibration that accounts for the differences in two optical components manufactured at different times, or by different manufacturers. The calibration preferably accounts for component specific characteristics.

One skilled in the art will appreciate that the controller can use a number of mechanisms to determine the ambient temperature in which the tester is operating. As the optical components may function differently under different operating conditions, the controller can effect temperature sensitive compensation for the components. As the optical components age, the temperature sensitivity may change. This drift in component characteristics is preferably compensated for by the controller, during periodic component recalibration, which is used to update the model of the components that is used in the determination of the transfer function.

The controller is preferably a specially developed controller, versions of which can be used by many manufacturers of devices aimed at similar or different species detection. One skilled in the art will appreciate that the tester of the present invention is a generalized control platform in the context of these biomedical measurements. Furthermore, one skilled in the art will readily appreciate that a preferred embodiment of the tester is governed by the ANSI standards for permissible exposure of the eye to wavelengths of light, for long and for short periods. The device is able to operate within allowable limits and contains built-in safeguards to protect users against overexposure.

Additional uses for the tester, related to the analysis of dissolved species, include, but are not limited to, use as an oximeter for the measurement of dissolved O₂ in the bloodstream which is commonly required after a suspected heart attack, use as a roadside alcohol and drug tester for law enforcement personnel, testers for determining that the drugs administered prior to surgery have been sufficiently dissolved, and testers for determining the presence and the concentrations of prohibited substances in athletic competitions.

One skilled in the art will appreciate that the tester of the present invention can be utilised to scan for more than one species dissolved in a subject's blood. This feature can be employed to test for related species that may interact with each other, such as a combination of glucose and alcohol in a diabetic subject. Such a tester can provide both single species information, in addition to the combination levels detected. The detection of a second species may be triggered by the concentration of the first species exceeding a defined threshold.

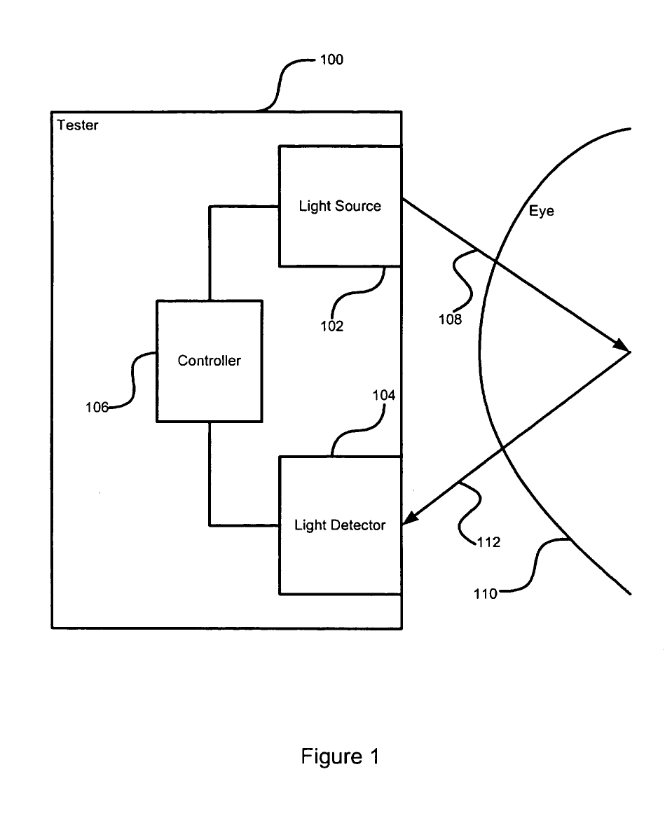

FIG. 1 illustrates a simplified schematic of a tester of the present invention. Tester 100 includes light source 102, light detector 104 and controller 106. Light source 102 transmits an incident light pulse 108 into eye 110, where it passes through blood, and is returned as modified light pulse 112. The modified light pulse is preferably returned after interaction with the ocular vasculature. Modified light pulse 112 is received and characterized by light detector 104. Controller 106 controls light source 102 to transmit incident light pulse 108 with at least one known characteristic. Light detector 106 provides the characterization of modified light pulse 112 to controller 106. The characterization of light pulse 106 may be optionally controlled so that only a small set of characteristics are characterized. Controller 106 determines a transfer function relating the known characteristic of light pulse 108 to the characterization of light pulse 112. Using a predetermined mapping function, controller 106 maps the transfer function to as species concentration level.

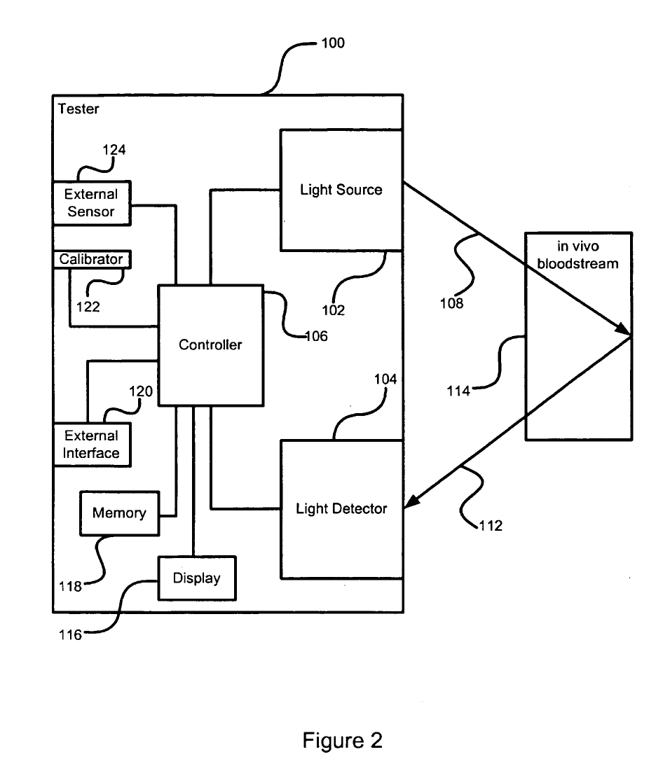

FIG. 2 illustrates an alternate embodiment of the present invention. Tester 100 still has light source 102 which generates light pulse 108, light detector 104 which receives modified light pulse 112, and controller 106. In addition, tester 100 includes a display 116 for providing a species concentration reading to the operator in readable form; storage memory 118 for storing a historical data set containing an archive of species concentration levels, and optionally time and date information for each reading; external interface 120 for interacting with external systems such as personal computers or medical equipment; and a calibrator 122 for providing calibration information from which controller 106 determines the mapping function between transfer functions and species concentrations; and external sensors, which may optionally include temperature sensors. The components shown in FIG. 2 can be provided independently of each other, though they are shown together for the sake of simplicity. Tester 110 is shown in FIG. 2 interacting with an in vivo bloodstream, which is not necessarily the ocular vasculature. As described above the ocular vasculature is a preferred target for the incident light pulse 108, due largely to its ability to return light, however, one skilled in the art will appreciate that any portion of the blood stream can be used, so long as there is sufficient returned light for light detector 104 to receive and characterize.

Display 116 preferably provides the operator of tester 100 with the species concentration levels as well as operational instructions and error message if necessary. In one embodiment display 116 is a simple liquid crystal display for displaying status messages and for providing species concentration levels.

Memory 118 serves as storage to store a historical archive of species concentration levels as they are obtained from the subject. This allows tester 100 to analyse or provide historical trends that may be useful in determining treatment for a condition such as diabetes.

External interface 120 provides a data transfer mechanism to a remote station such as a personal computer or a piece of medical equipment. The external interface preferably utilizes standard off the shelf connectivity components such as an IEEE 802.11 interface, a Bluetooth™ interface, a Universal Serial Bus interface, or an Ethernet Networking interface. These connections allow the device readings to be provided to another system, either for backup or for further analysis.

Calibrator 122 provides controller 106 with a predetermined mapping function, by creating a correlation between transfer functions determined by the controller, and species concentration levels determined by another mechanism. In the case of a glucose monitor, the calibrator may be a standard blood tester integrated for convenience. In another embodiment, the calibrator 122 is integrated with the external interface 120, so that externally determined readings are provided through external interface 120 to controller 106. In a further embodiment, ocular readings are taken by tester 100, and the user provides manual input of the species concentration using a keypad interface. Other calibration mechanisms will be well understood by those skilled in the art.

External sensor 124 is employed to obtain operational information about tester 100. As it is known that optical components such as light source 102 and light detector 104 are temperature sensitive, it may be advantageous to employ a temperature sensor as external sensor 124 so provide accurate calibration of the device as temperature changes. Other such external or environmental effects can be determined through the use of an appropriate external sensor 124, and compensated for by controller 106.

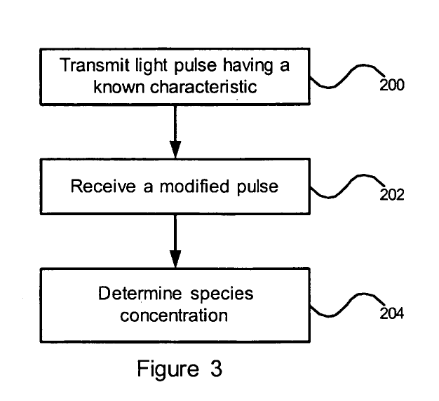

FIG. 3 is a flowchart illustrating a method of the present invention. In step 200 a light pulse having a known characteristic is generated and transmitted. In step 202 a modified light pulse corresponding to the transmitted light pulse is received. The modified light pulse varies from the transmitted light pulse in certain manners as a result of its interaction with the dissolved species. The species concentration is determined in step 204 based on the relationship between the transmitted light pulse and the received light pulse.

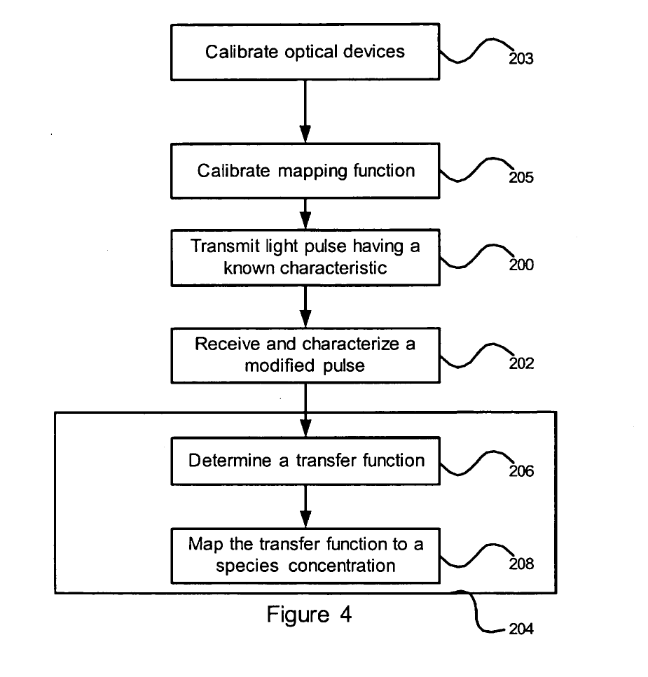

FIG. 4 is a flowchart illustrating a more detailed method of the present invention. The method illustrated in FIG. 4 begins with a calibration process in steps 203 and 205. In step 203, the optical components used to generate the light pulse and receive the light pulse are calibrated. This calibration allows for confidence that the characteristics of the transmitted pulse are known, and that the determined characteristics of the received pulse are accurately determined. In step 205 a mapping function used to map transfer functions to species concentrations is performed. This calibration process provides a set of baseline readings that can be used to map the transfer function to a species concentration. As before, in steps 200 and 202, a light pulse is transmitted, and a modified light pulse is received. Step 204, the determination of the species concentration includes steps 206 and 208. In step 206 a transfer function relating the at least one known characteristic to at least one characteristic of the modified pulse is determined. A transfer function relating a plurality of known characteristics of the transmitted light pulse to a plurality of corresponding characteristics in the modified pulse can also be determined for greater accuracy in the subsequent step. In step 208 the determined transfer function is mapped to a species concentration using a mapping function. The mapping function is preferably determined in accordance with calibration data.

One skilled in the art will appreciate that that the method and tester disclosed above can be used in the detection of a species in a blood vessel, as well as in the detection of particular species levels in a blood vessel. Though much of the above discussion has related to the detection of glucose, alcohol and THC, other species, including lactose, heparin, iodine, potassium, iron, cyclosporine, cholesterol, nitrogen, other drugs, and various pathogens can be detected so long as they exhibit identifiable optical characteristics in the returned light pulse. Additionally, one skilled in the art will appreciate that the tester can be utilised in veterinary settings, for the detection of species in various different animals. The tester would preferably be calibrated to the particular animal, or possibly the breed. Such a tester could be used to monitor canine diabetes, or to detect medicine concentration levels in a pet. Such a tester could also be employed to detect pathogens in livestock prior to butchering, or to possibly detect elevated hormone levels indicative of various conditions. The levels of a plurality of species in a subject's reading may additionally find use in a biometric identification system.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. A blood species tester, for determining the concentration of a species flowing in a blood vessel of a subject, the tester comprising:
a light source for transmitting a light pulse, having at least one known characteristic, to an eye of the subject;
a light detector for receiving and characterizing a modified light pulse; and
a controller, for determining a transfer function relating at least one of the at least one known characteristics to at least one characteristic of the modified pulse, and for mapping the determined transfer function to a species concentration in accordance with a predetermined mapping function.

2. The tester of claim 1, wherein the species is selected from a list including glucose, alcohol, and tetrahydrocannabinol.

3. The tester of claim 1, wherein the at least one known characteristic is selected from a list including wavelength and intensity.

4. The tester of claim 1, wherein the at least one characteristic of the modified pulse is selected from a list including intensity, polarisation, and fluorescence.

5. The tester of claim 1 wherein the light source includes means for transmitting a pulse having a plurality of known characteristics.

6. The tester of claim 5, wherein the means for transmitting a plurality of known characteristics includes a plurality of light sources, each of the plurality for transmitting a light pulse, having at least one known characteristic.

7. The tester of claim 5, wherein the controller determines transfer functions relating each of the known characteristics to measured characteristics in the modified pulse.

8. The tester of claim 1, wherein the controller includes a memory means for storing historical species concentration information.

9. The tester of claim 8, wherein the controller includes subject identification means for obtaining subject identification information from at least one characteristic in the modified light pulse, for storing identification information with associated historical species concentration information in the memory means, and for differentiating species concentration information stored in the memory on the basis of the associated identification information.

10. The tester of claim I including a calibration mechanism for determining the mapping function by correlating a plurality of determined transfer functions to species concentrations obtained using an external species concentration tester.

11. A method of determining a species concentration in a subject's blood vessels, the method comprising:
generating and transmitting a light pulse having at least one known characteristic;
receiving and characterizing a modified, returned light pulse; and
determining the species concentration in accordance with a mapping function and a transfer function relating the at least one known characteristic with a corresponding characteristic in the modified, returned light pulse.

12. The method of claim 11 including the step of calibrating the mapping function to map the transfer function to known species concentrations in advance of the step of determining the species concentration.

13. The method of claim 11, wherein the step of generating and transmitting includes transmitting the light pulse into an eye of the subject.

14. The method of claim 13, wherein the step of receiving and characterizing includes receiving the modified returned light pulse from the ocular vasculature.

15. The method of claim 11, wherein the step of generating and transmitting includes transmitting a light pulse having a plurality of known characteristics.

16. The method of claim 11, wherein the step of generating and transmitting includes transmitting a series of light pulses.

17. The method of claim 16, wherein the step of transmitting a series of light pulses includes generating and transmitting a series of light pulses, each of the series of light pulses having at least one known characteristic, at least two of the series of light pulses having different known characteristics.

18. The method of claim 16, wherein the step of receiving and characterizing, includes receiving and characterizing a plurality of modified returned light pulses, and wherein the step of determining the species concentration includes determining a transfer function relating the known characteristic of each light pulse to a corresponding characteristic in a corresponding returned modified pulse.

19. The method of claim 11, wherein the step of determining the species concentration includes determining the transfer function in accordance with external environmental factors.

20. The method of claim 19, wherein the external environmental factors include ambient temperature.

21. The method of claim 11 further including the step of recording the determined species concentration in an archive of historical species concentrations.
